(19) United States
(12) Patent Application Publication
(10) Pub. No.: US 2006/0210535 A1
(43) Pub. Date: Sep. 21, 2006

(54) Means for regulating the expression of human isoforms of ant

(76) Inventors: Annie Borgne, Paris (FR); Dominique Rebouillat, Paris (FR); Etienne Jacotot, Paris (FR)

Correspondence Address: NIXON & VANDERHYE, PC, 901 NORTH GLEBE ROAD, 11TH FLOOR, ARLINGTON, VA, 22203, US

(21) Appl. No.: 10/542,708

(22) PCT Filed: Jan. 21, 2004
(86) PCT No.: PCT/FR04/00127

§ 371 Date: Nov. 10, 2005

(30) Foreign Application Priority Data
Jan. 21, 2003 (FR) 0300622

(51) Int. Cl. A61K 48/00; C07H 21/02

(52) U.S. Cl. 424/93.2; 514/44; 536/23.2

(57) ABSTRACT

ARNi which can selectively inhibit expression of an isoform of ANT, characterized in that said ARNi are an ARN duplex, one of the strands being highly homologous to a fragment of ARNm coding for said isoform of ANT.

The invention relates to means for regulating the expression of human isoforms of ANT, more particularly to interfering RNA (iRNA) duplexes and uses thereof for said regulation, and to the uses of the cDNAs encoding the isoforms.

The adenine nucleotide translocator (ANT) is the most abundant protein of the inner membrane of mitochondria. ANT has two distinct functions: it is, firstly, responsible for the transport of adenine nucleotides across the inner mitochondrial membrane (import of ADP for oxidative phosphorylation; export of ATP to the cytosol for general metabolism). Secondly, ANT plays an essential role during the mitochondrial phase of apoptosis. This is because ANT can adopt a nonspecific pore conformation, which results in permeabilization of mitochondrial membranes and in the triggering of cell death (Kroemer & Reed 2000).

The genes encoding ANTs have been cloned in a large number of species, such as yeast, various plants, cows, rats, mice and humans. All these species have several isoforms, and the structure of the genes is highly conserved, with an organization consisting of 4 exons separated by 3 introns. Human ANT exists in three isoforms (ANT1, ANT2 and ANT3) encoded by three different nuclear genes, which have been cloned and sequenced. ANT1 (chromosome 4) is mainly expressed in the heart and the skeletal muscles. A hereditary disease in humans, associated with a mutation in ANT1 (substitution of alanine 114 to proline), is known. This disease is progressive external ophthalmoplegia (a rare condition characterized by substantial deletions of the mitochondrial DNA). ANT2 (X chromosome) is very weakly expressed in mature tissues. The highest expression levels for ANT2 are observed in proliferating cells such as myoblasts and tumor cells. ANT2 is also specifically found in cells transformed with the SV40 virus, and also the lines devoid of mitochondrial DNA (rho°). ANT3 (pseudoautosomal region of the X and Y chromosomes) is expressed ubiquitously in all differentiated tissues.

Apoptosis is a process of cell suicide that takes place in three phases: a pre-mitochondrial phase (heterogeneous), a mitochondrial phase (decision to die), and a degradation phase (“putrefaction” of the cell). ANT, a protein inserted into the inner mitochondrial membrane, has the ability to form a pore which radically changes the role of the mitochondrion: when ANT is in its OPEN PORE state, the mitochondrion becomes a cell-destruction organ.

The following points have today been established:
It is possible to kill cells in vitro by inducing the pore function of ANT (Belzac, Jacotot et al., Cancer Res. 2001 Feb. 15. 61(4):1260-4).
It is possible to protect cardiac cells ex vivo (isolated reperfused heart) by blocking the pore function of ANT (Di Lisa et al., J Biol Chem. 2000 Nov 9).
It is possible to protect neurons in vivo against death subsequent to cerebral ischemia, by inhibiting ANT (Cao et al., J Cereb Blood Flow Metab. 2001 April 21(4):321-333).

ANT is therefore a major control point for apoptosis and is regulated by endogenous proteins such as the Bax (pro-apoptotic) tumor suppressor and the Bcl-2 (anti-apoptotic) oncoprotein. ANT is also regulated by viral proteins such as Vpr (pro-apoptotic derived from HIV) and vMIA (anti-apoptotic derived from CMV). It is therefore an ideal target for combating pathological deregulation of apoptosis.

Recent data have revealed that double-stranded RNA (dsRNA) induces quenching of the expression of genes whose sequence is very homologous to the sequence of one of the two strands of RNA of the duplex. This phenomenon, called RNA interference or iRNA, results in degradation of the messenger RNAs (Hammond et al., 2001, Sharp, 2001). Tuschl et al. have demonstrated that the introduction into mammalian cells of a 21-nucleotide RNA duplex (small interfering RNA or siRNA) results in the specific inhibition of gene expression (Elbashir et al., 2001). After transfection, the siRNAs act hand in hand with cellular components (the DICER enzyme and the RISC complex) in order to abolish expression of the target gene.

The inventors have noted that it is possible to regulate apoptosis for therapeutic purposes by acting on the level of expression of the human isoforms of ANT in a selective manner.

In particular, it has been found that iRNAs designed from defined 21-nucleotide regions of the coding sequence of each ANT isoform makes it possible to develop duplex iRNAs capable, after transfection, of selectively abolishing the expression of each isoform.

The aim of the invention is therefore to provide novel products which, when combined with any method for transferring nucleic acids, can be used in human and animal therapy.

The invention is directed toward iRNAs capable of selectively inhibiting the expression of an ANT isoform, characterized in that said iRNAs are an RNA duplex, one of the strands being highly homologous to a fragment of the mRNA encoding said ANT isoform.

Advantageously, the iRNAs of the invention are siRNAs (small interfering RNAs) of 18 to 25 nucleotides, more particularly of 21 nucleotides.

Preferred iRNAs are chosen from the duplexes with strands of sequences SEQ ID No. 1 and SEQ ID No. 2; SEQ ID No. 3 and SEQ ID No. 4; SEQ ID No. 5 and SEQ ID No. 6:

| | |
|---|---|
| SEQ ID No. 1: | 5′-acagaucagugcugagaagdTdT-3′ |
| SEQ ID No. 2: | 5′-cuucucagcacugaucugudTdT-3′ |
| SEQ ID No. 3: | 5′-gcagaucacugcagauaagdTdT-3′ |
| SEQ ID No. 4: | 5′-cuuaucugcagugaucugcdTdT-3′ |
| SEQ ID No. 5: | 5′-gggcaucguggacugcauudTdT-3′ |
| SEQ ID No. 6: | 5′-aaugcaguccacgaugcccdTdT-3′ |

The invention is also directed toward constructs containing at least one iRNA as defined above or DNA sequences encoding each of the strands of these iRNAs.

In one embodiment of the invention, the construct is characterized in that the iRNA is associated with a vector that facilitates its administration, its passage across membranes, tissues or biological integuments, in particular cytoplasmic membranes, mitochondrial membranes, nuclear membranes, skin, mucous membranes, endothelial walls, the blood-brain barrier, and also its bioavailability, its stability and its pharmacodistribution, such as a peptide, a liposome, nanoparticles (nanospheres, nanotubes), or a non-natural oligomer such as urea polymers.

In another embodiment, the construct is characterized in that the iRNA is associated with a vector for transferring nucleic acids, such as retroviruses (Barton and Medzhitov, PNAS, 2002, vol. 99 (23): p 14943-14945), transposons, adenoviruses (Xia et al.; Nature Bidech, 2002, vol. 20, p 1005-1010) or plasmids (Brummelkamp et al., Cancel Call, 2002, p 243-247).

The invention is also directed toward the pharmaceutical compositions characterized in that they contain an effective amount of at least one iRNA as defined above, or a construct as defined above, in combination with a pharmaceutically acceptable vehicle.

Advantageous pharmaceutical compositions are characterized in that they are in injectable form.

Other presentation forms are suitable for oral, parenteral, rectal or topical administration (Levis et al., Nature Genetics, 2002, vol. 32, p 107-108).

The iRNAs, constructs or pharmaceutical compositions as defined above are characterized in that they have the ability to regulate (to induce or to inhibit) mitochondrial membrane permeabilization and cell death of apoptotic, necrotic and autophagic type and related mechanisms.

The compositions of the invention make it possible to regulate the expression of human isoforms of ANT and, in this respect, are particularly useful for the treatment of pathologies associated with deregulation of apoptosis and other related forms of cell death.

The invention therefore relates, in part, to the use of siRNA-ANT1, siRNA-ANT2 and/or siRNA-ANT3 for inducing/promoting (siRNA-ANT2) or, conversely inhibiting (siRNA-ANT and/or siRNA-ANT3) the drop in mitochondrial transmembrane potential (ΔΨm) and apoptosis and death of apoptotic, necrotic and autophagic type, and related mechanisms.

The invention therefore also relates to the use of hANT1, hANT2 and/or hANT3 cDNAs for inducing/promoting (hANT1 cDNA and/or hANT3 cDNA) or, conversely, inhibiting (hANT2 cDNA) the drop in mitochondrial transmembrane potential (ΔΨm) and apoptosis.

Mention is in particular made of their use for treating an apoptosis deficiency, for example in the various forms of cancer, and autoimmune diseases, such as disseminated lupus erythematosus or arthritis.

In other uses, these compositions are used for treating an excess of apoptosis, such as, for example, neurodegenerative diseases (Alzheimer's disease, Parkinson's disease, Huntington's disease) and cerebral and cardiac ischemias.

For example, ANT1 or ANT3 siRNAs, or alternatively ANT2 cDNA, may be used for inhibiting neuronal death in ischemic situations or situations of neurodegenerative pathologies, or else for inhibiting cardiomyocyte death in ischemic situations, or hepatocyte death (viral infections, drug-related poisonings). For example, h-ANT2 siRNAs and/or h-ANT1 or h-ANT3 cDNAs may be used for inducing tumor cell apoptosis or autoreactive lymphocyte apoptosis.

Said pharmaceutical compositions are also of great advantage for the treatment of HIV infections.

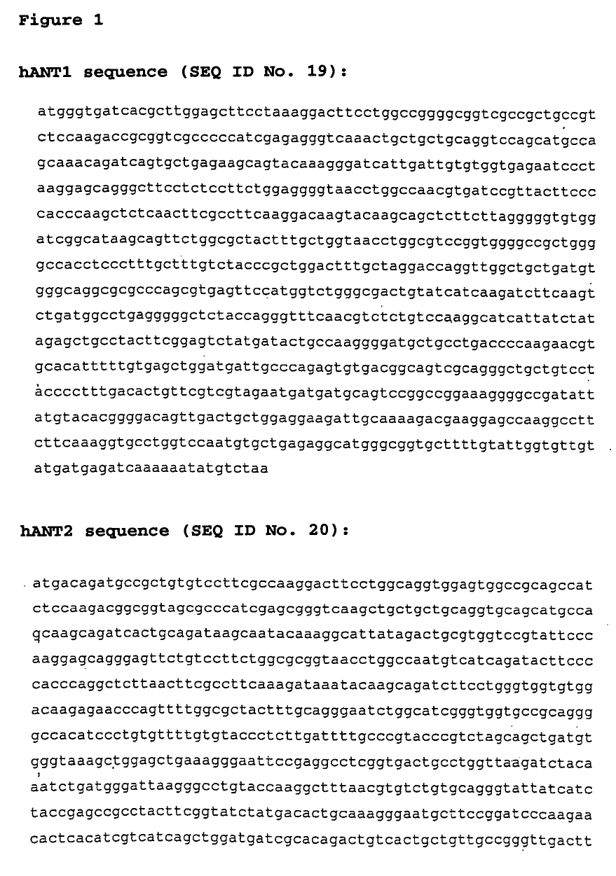

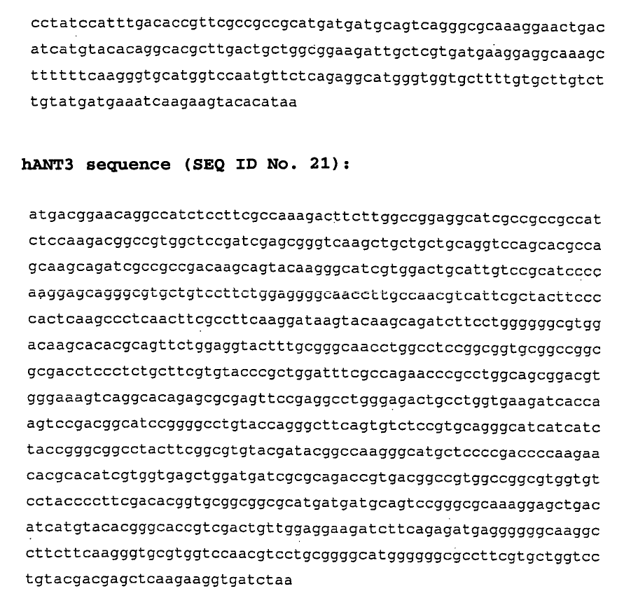

Other characteristics and advantages of the invention will emerge in the subsequent description, and with reference to FIGS. 1 to 6, which represent, respectively:

FIG. 1. Complete cDNA sequences encoding the three human isoforms of ANT, isolated after RT/PCR using RNAs originating from 293T and HeLa cells.

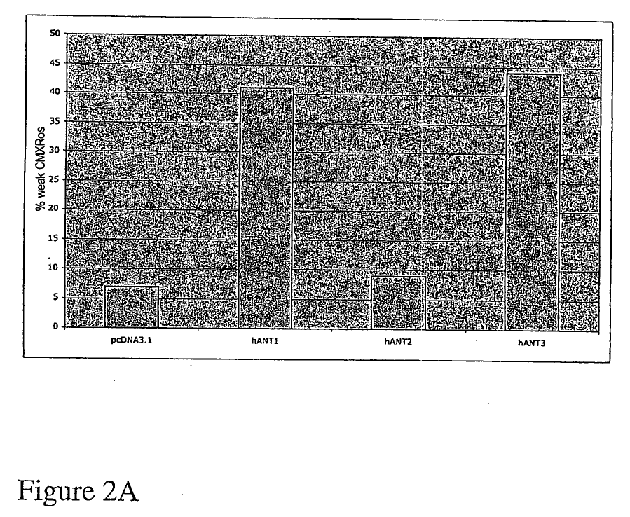

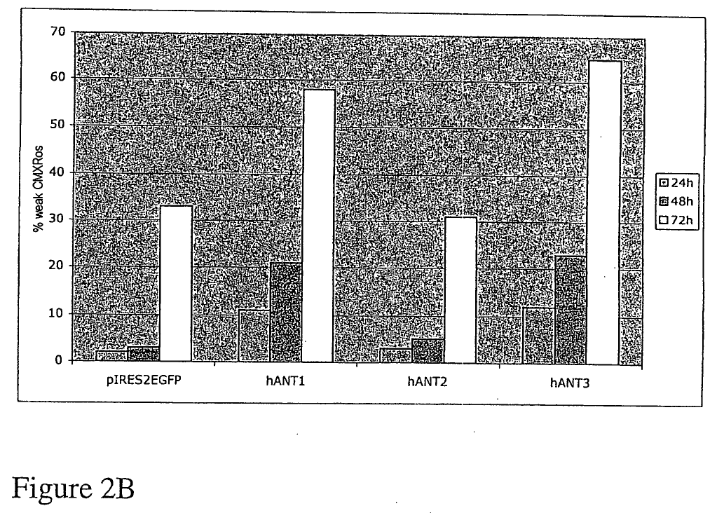

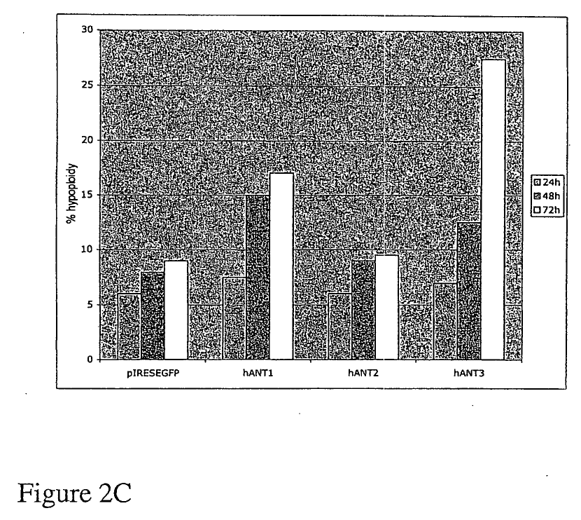

FIG. 2. Expression of the hANT1 isoform and expression of the hANT3 isoform induce apoptosis. Flow cytometry analysis of 293T cells, 24 hours after cotransfection of 1 μg of vector pIRES-2-eGFP with 1 μg of vector pcDNA3.1-hANT. The intensity of the CMXRos label is quantified only on the GFP positive cells. B. Flow cytometry analysis of 293T cells, 24, 48 or 72 hours after transfection with 1 μg of vector pIRES-2-eGFP or with 1 μg of each vector pIRES-eGFP-hANT. The intensity of the CMXRos label is quantified only on the GFP positive cells. C. Flow cytometry analysis of the frequency of hypoploid nuclei on 293T cells, 24, 48 or 72 hours after transfection with 1 μg of vector pIRES-eGFP or 1 μg of each vector pIRES-eGFP-hANT.

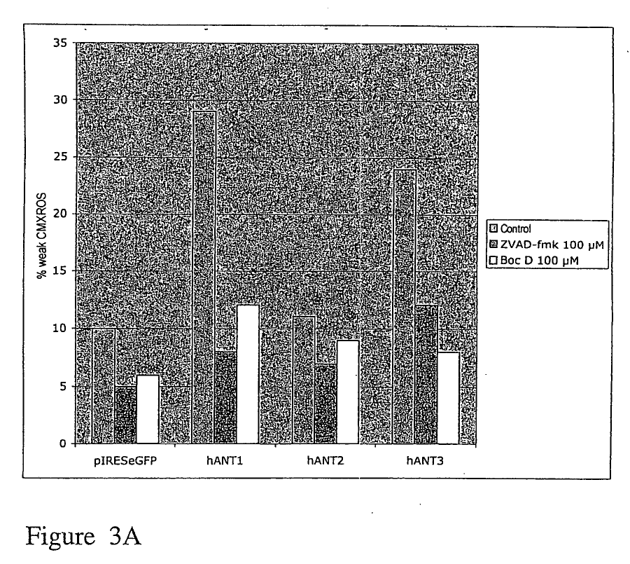

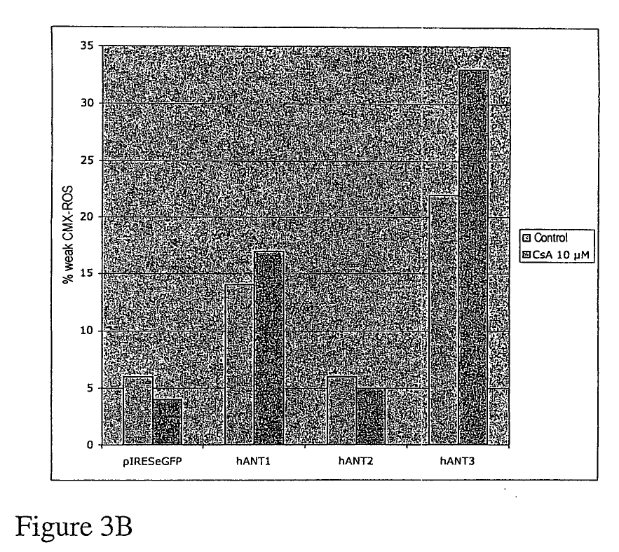

FIG. 3. The apoptosis induced by the expression of hANT1 and hANT3 is inhibited by ZVAD and Boc D but not by CsA. A. Flow cytometry analysis of 293T cells 48 hours after transfection with 1 μg of vector pIRES-2-eGFP or with 1 μg of each vector pIRES-eGFP-hANT in the presence or absence of 10 μM of CsA. The intensity of the CMXRos label is quantified only on the GFP positive cells. B. Flow cytometry analysis of 293T cells 48 hours after transfection with 1 μg of vector pIRES-2-eGFP or with 1 μg of each vector pIRES-eGFP-hANT in the presence or absence of 100 μM of ZVAD-fmk or of 100 μM of Boc D. The intensity of the CMXRos label is quantified only on the GFP positive cells.

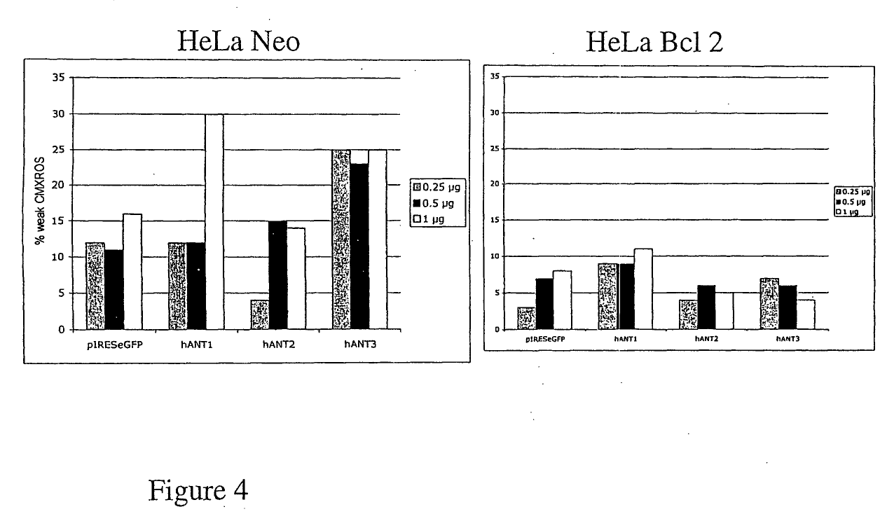

FIG. 4. The expression of Bcl2 inhibits apoptosis induced by the expression of the hANT1 and hANT3 isoforms. HeLa Neo and Bcl2 cells are transfected with 1 μg of vector pIRES-2-eGFP or with 1 μg of each vector pIRES-eGFP-hANT and, after 72 hours, the intensity of the CMXRos label is analyzed by flow cytometry on the GFP positive cells.

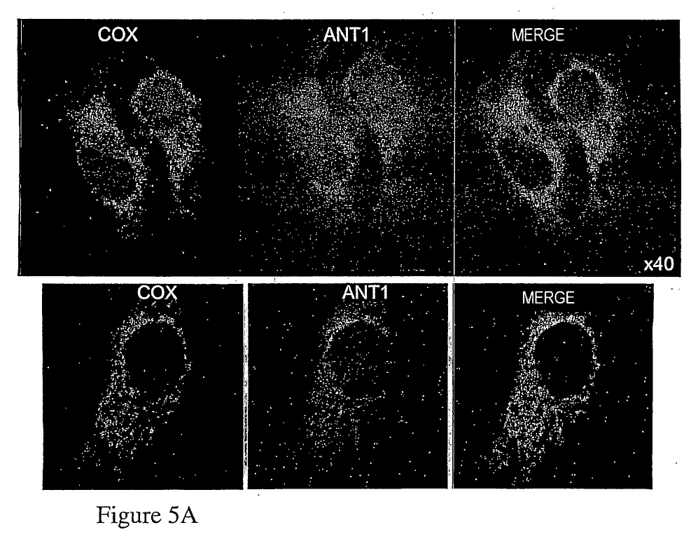

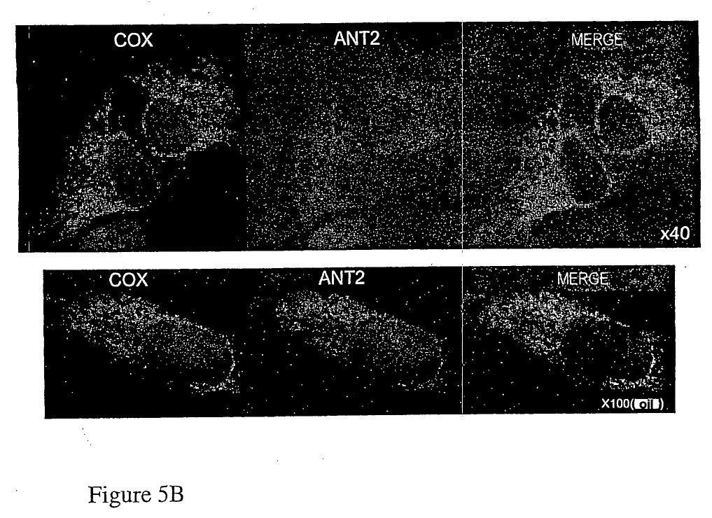

FIG. 5. Subcellular localization of the hANT1 and hANT2 isoforms. HeLa cells are transfected with 1 μg of vector pcDNA3.1V5-hANT1 (A) or with 1 μg of vector pcDNA3.1V5-hANT2 (B) and then fixed with paraformaldehyde. The colocalization of the hANT-V5 fusion proteins with the COX mitochondrial protein is determined by immunofluorescent detection of the V5 epitope (green fluorescence) and of the COX protein (red florescence). The “merge” image represents the superimposition of the green fluorescence and red fluorescence showing the colocalization.

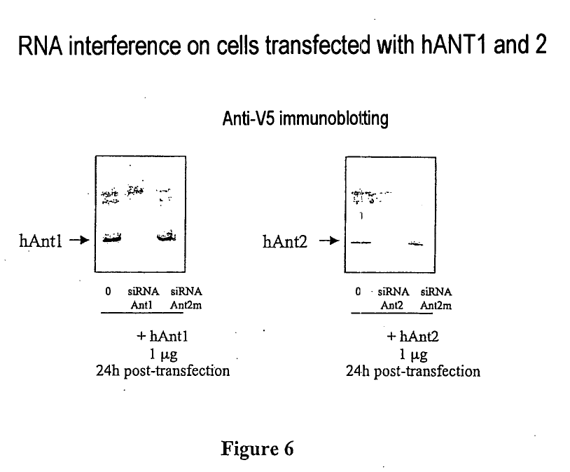

FIG. 6. Specific inhibition of the expression of the human isoforms 1 and 2 of ANT via the use of specific siRNAs.
(A) HeLa cells are cotransfected with, firstly, an expression vector pcDNA3.1V5-hANT1 and, secondly, siRNAs specific for hANT1 or hANT2mut.
(B) HeLa cells are cotransfected with, firstly, an expression vector pcDNA3.1V5-hANT2 and, secondly, siRNAs specific for hANT2 or hANT2mut.

24 hours after transfection, the cells are lyzed and the expression of the ANT isoforms is determined by Western blotting using an anti-V5 monoclonal antibody.

Cotransfections: HeLa cells are cultured in 6-well plates in DMEM/Glutamax-I supplemented with 10% of fetal calf serum. After 24 hours, the cells are transfected by adding 3 μl of lipofectamine 2000 (Invitrogen), 3 μg of siRNA and 1 μg of vector pcDNA3.1V5-hANT1 or 2 in serum-free DMEM (final volume of 500 μl). The cells are rinsed 6 hours after transfection and maintained in culture for 24, 48 or 72 hours.

Cell extract preparations and Western blotting: The cells are resuspended in 100 μl of lysis buffer (25 mM Tris-HCl, pH 7.5, 25 mM NaCl, 5 mM EDTA, 1% Triton X-100, cocktail of protease inhibitors) and centrifuged for 10 minutes at 13 000 rpm at 4° C. 10 μl of the supernatant are collected in order to carry out a Bradford test. The extracts are then analyzed by SDS-PAGE gel after denaturation for 3 minutes at 100° C. in the presence of SDS-Laemmli buffer. After transfer, the proteins are revealed with an anti-V5 antibody (1/5000 Invitrogen).

Cloning of the human isoforms of ANT and production of expression vectors: Total RNA from 293T cells and from HeLa cells was isolated (Trizol protocol) and used in reverse transcription/amplification experiments initiated with an oligo dT-type primer. Primers specific for the human isoforms of ANT (hANT1, hANT2 and hANT3) were synthesized based on the sequences published in GenBank in order to specifically amplify the complete cDNA of each of the isoforms (table 1). These products were then subcloned into the vector pGEM-T after the addition of a dAdenosine residue at their ends. The sequence of each insert was verified (FIG. 1). The cDNAs encoding the three isoforms were then cloned into expression vectors: pcDNA3.1 (version +, Invitrogen) and pIRES-2-eGFP (Clontech). To generate fusion proteins with the V5 epitope corresponding to the three isoforms, an amplification approach (table 2) made it possible to modify the ends of the cDNAs encoding the three isoforms (mutation of the STOP codon and also addition of restriction enzyme recognition sequences) and to subclone these products into the vector pcDNA3.1-V5 (version A, Invitrogen). The final constructs were verified by sequencing.

Apoptotic potential of the human isoforms of ANT: The transfection experiments were carried out on 293T cells, using the empty vector pIRES-2-GFP as a control or the vectors pIRES-2-eGFP containing the sequences of the cDNAs encoding the three isoforms of hANT. At a given time post-transfection, the cells were analyzed by flow cytometry.

The results show that the expression of the hANT1 and hANT3 isoforms results in a dissipation of the mitochondrial potential, thus triggering apoptosis, whereas the expression of the hANT2 isoform does not affect the mitochondrial integrity (FIG. 2).

Using a similar experimental approach, we demonstrate that the apoptosis associated with the expression of the hANT1 and hANT3 isoforms is inhibited by caspase inhibitors (ZVAD and Boc D) (FIG. 3A) but not by cyclosporin A (CsA) (FIG. 3B).

We also demonstrate, using HeLa cells overexpressing the Bcl2 protein, that the latter is capable of inhibiting the apoptosis induced by the hANT1 and hANT2 isoforms (FIG. 4).

Subcellular localization of the hANT1 and hANT2 isoforms: After transfection of HeLa cells with constructs encoding the hANT1-V5 and hANT2-V5 fusion proteins, we carried out immunolabeling in order to determine the subcellular localization of hANT1 and hANT2. The analysis of the localization of the signal obtained with an anti-V5 antibody and the signal obtained with an antibody directed against COX (a mitochondrial protein) demonstrates a mitochondrial localization for the hANT1 and 2 isoforms (FIG. 5).

iRNA Duplex of the Human Isoforms of ANT

Preparation of iRNAs. The double-stranded siRNAs corresponding to the cDNA sequences of human ANT1 (AAACAGATCAGTGCTGAGAAG, nucleotides 127-147), human ANT2 (AAGCAGATCACTGCAGATAAG, nucleotides 127-147), human ANT2 containing four mutations (AAGCGGATCGCTACAAATAAG, nucleotides 127-147) and human ANT3 (AAGGGCATCGTGGACTGCATT, nucleotides 154-174) were designed according to the recommendations of Elbashir et al. (2001). The duplexes were prepared by Proligo (France).

| | |
|---|---|
| hANT1 (127-147) | |
| DNA sequence: | |
| 5′-aaacagatcagtgctgagaag-3′ | (SEQ ID No. 7) |
| iRNA duplex: | |
| 5′-acagaucagugcugagaagdTdT-3′ | (SEQ ID No. 8) |
| 5′-cuucucagcacugaucugudTdT-3′ | (SEQ ID No. 9) |
| hANT2 (127-147) | |
| DNA sequence: | |
| 5′-aagcagatcactgcagataag-3′ | (SEQ ID No. 10) |
| iRNA duplex: | |
| 5′-gcagaucacugcagauaagdTdT-3′ | (SEQ ID No. 11) |
| 5′-cuuaucugcagugaucugcdTdT-3′ | (SEQ ID No. 12) |
| hANT2mut (127-147) | |
| DNA sequence: | |
| 5′-aagcggatcgctacaaataag-3′ | (SEQ ID No. 13) |
| iRNA duplex: | |
| 5′-gcggaucgcuacaaauaagdTdT-3′ | (SEQ ID No. 14) |
| 5′-cuuauuuguagcgauccgcdTdT-3′ | (SEQ ID No. 15) |
| hANT3 (154-174) | |
| DNA sequence: | |
| 5′-aagggcatcgtggactgcatt-3′ | (SEQ ID No. 16) |
| iRNA duplex: | |
| 5′-gggcaucguggacugcauudTdT-3′ | (SEQ ID NO. 17) |
| 5′-aaugcaguccacgaugcccdTdT-3′. | (SEQ ID No. 18) |

The tables hereinafter give, respectively, the sequences of the primers used:
Table 1: in RT/PCR experiments in order to clone the cDNA encoding the three human isoforms of ANT.

Table 2. for the construction of the expression vectors containing the cDNAs encoding the hANT-V5 fusion proteins.

TABLE 1

| | |
|---|---|
| Sense primer | Antisense primer |

| | | | |
|---|---|---|---|
| hANT1 | (SEQ ID No. 22 and 23) | 5′ATGGGTGATCACGCTTGGAGCTTCCTAAAG3′ | 5′TTAGACATATTTTTTGATCTCATCATACAA3′ |
| hANT2 | (SEQ ID No. 24 and 25) | 5′ATGACAGATGCCGCTGTGTCCTTCGCCAAG3′ | 5′TTATGTGTACTTCTTGATTTCATCATACAA3′ |
| hANT3 | (SEQ ID No. 26 and 27) | 5′ATGACGGAACAGGCCATCTCCTTCGCCAAA3′ | 5′TTAGATCACCTTCTTGAGCTCGTCGTACAG3′ |

TABLE 2

| | |
|---|---|
| Sense primer | Antisense primer |

| | | | |
|---|---|---|---|
| hANT1 | 5′TAAGGTACCATGGGTGATCACGCTTGGA3′ | (SEQ ID No. 28 and 29) | 5′ATCTCGAGGACATATTTTTTGATCTC3′ |
| hANT2 | 5′TAAGGTACCATGACAGATGCCGCTGTGT3′ | (SEQ ID No. 30 and 31) | 5′ATCTCGAGTGTGTACTTCTTGATTTC3′ |
| hANT3 | 5′TAAGGTACCATGACGGAACAGGCCATCT3′ | (SEQ ID No. 32 and 33) | 5′ATCTCGTGGATCACCTTCTTGAGCTC3′ |

### REFERENCES

Hammond, S. M., Caudy, A. A. and Hannon, G. J. (2001). Post-transcriptional gene silencing by double-stranded RNA. Nat Rev Genet, 2, 110-119.
Sharp, P. A. (2001). RNA interference-2001. Genes Dev. 15, 485-490.
Elbashir, S. M., Harborth, J., Lendeckel, W., Yalcin, A., Weber, K. and Tuschl, T. (2001). Duplexes of 21-nucleotide RNAs mediate RNA interference in cultured mammalian cells, Nature, 411, 494-498.

1. An iRNA capable of selectively inhibiting the expression of an ANT isoform, characterized in that said iRNA is an RNA duplex, one of the strands being highly homologous to a fragment of the mRNA encoding said ANT isoform.

2. The iRNA as claimed in claim 1, characterized in that it is an siRNA of 18 to 25 nucleotides, more particularly of 21 nucleotides.

3. The iRNA as claimed in claim 2, characterized in that it has the sequence SEQ ID No. 1, SEQ ID No. 2 or SEQ ID No. 3.

4. A construct containing at least one iRNA as claimed in claim 1, or DNA sequences encoding each of the strands of these iRNAs.

5. The construct as claimed in claim 4, characterized in that the iRNA is associated with a vector that facilitates its administration, its passage across membranes, tissues or biological integuments, in particular cytoplasmic membranes, mitochondrial membranes, nuclear membranes, skin, mucous membranes, endothelial walls, the blood-brain barrier, and also its bioavailability, its stability and its pharmacodistribution, such as a peptide, a liposome, nanoparticles (nanospheres, nanotubes), or a non-natural oligomer such as urea oligomers.

6. The construct as claimed in claim 4, characterized in that the vectors are vectors for transferring nucleic acids, such as retroviruses, transposons, adenoviruses or plasmids.

7. A pharmaceutical composition characterized in that it contains an effective amount of at least one iRNA as claimed in claim 1, or a construct containing at least one of said iRNA or DNA sequences encoding each of the strands of these iRNAS, in combination with a pharmaceutically acceptable vehicle.

8. The pharmaceutical composition as claimed in claim 7, characterized in that it is in injectable form, or in a form that can be administered orally, parenterally, rectally or topically.

9. The iRNA as claimed in claim 1, or a construct containing at least one of said iRNA or DNA sequences encoding each of the strands of these iRNAS, or a pharmaceutical composition containing an effective amount of at least one of the iRNA, characterized in that it has the ability to regulate (to induce or to inhibit) mitochondrial membrane permeabilization and cell death of apoptotic, necrotic and autophagic type, and related mechanisms.


SEQUENCE LISTING (33 sequences)

SEQ ID NO: 1 - RNA, 21 nt, Artificial sequence
  Human
acagaucagugcugagaagnn

SEQ ID NO: 2 - RNA, 21 nt, Artificial sequence
  Human
cuucucagcacugaucugunn

SEQ ID NO: 3 - RNA, 21 nt, Artificial sequence
  Human
gcagaucacugcagauaagnn

SEQ ID NO: 4 - RNA, 21 nt, Artificial sequence
  Human
cuuaucugcagugaucugcnn

SEQ ID NO: 5 - RNA, 21 nt, Artificial sequence
  Human
gggcaucguggacugcauunn

SEQ ID NO: 6 - RNA, 21 nt, Artificial sequence
  Human
aaugcaguccacgaugcccnn

SEQ ID NO: 7 - DNA, 21 bp, Artificial sequence
  Human
aaacagatcagtgctgagaag

SEQ ID NO: 8 - DNA, 21 bp, Artificial sequence
  Human
aagcagatcactgcagataag

SEQ ID NO: 9 - DNA, 21 bp, Artificial sequence
  Human
aagcggatcgctacaaataag

SEQ ID NO: 10 - DNA, 21 bp, Artificial sequence
  Human
aagggcatcgtggactgcatt

SEQ ID NO: 11 - RNA, 21 nt, Artificial sequence
  Human
acagaucagugcugagaagnn

SEQ ID NO: 12 - RNA, 21 nt, Artificial sequence
  Human
cuucucagcacugaucugunn

SEQ ID NO: 13 - RNA, 21 nt, Artificial sequence
  Human
gcagaucacugcagauaagnn

SEQ ID NO: 14 - RNA, 21 nt, Artificial sequence
  Human
cuuaucugcagugaucugcnn

SEQ ID NO: 15 - RNA, 21 nt, Artificial sequence
  Human
gcggaucgcuacaaauaagnn

SEQ ID NO: 16 - RNA, 21 nt, Artificial sequence
  Human
cuuauuuguagcgauccgcnn

SEQ ID NO: 17 - RNA, 21 nt, Artificial sequence
  Human
gggcaucguggacugcauunn

SEQ ID NO: 18 - RNA, 21 nt, Artificial sequence
  Human
aaugcaguccacgaugcccnn

SEQ ID NO: 19 - DNA, 893 bp, Artificial sequence
  Human
atgggtgatcacgcttggagcttcctaaaggacttcctggccggggcggtcgccgctgccgtctccaagaccgcggtcgcccccatcagagggtcaaactgctgctgcaggtccagcatgccagcaaacagatcagtgctgagaagcagtacaaagggatcattgattgtgtggtgagaatccctaaggagcagggcttcctctccttctggaggggtaacctggccaacgtgatccgttacttccccacccaagctctcaacttcgccttcaaggacaagtacaagcagctcttcttagggggtgtggatcggcataagcagttctggcgctactttgctggtaacctggcgtccggtggggccgctggggccacctccctttgctttgtctacccgctggactttgctaggaccaggttggctgctgatgtgggcaggcgcgcccagcgtgagttccatggtctgggcgactgtatcatcaagatcttcaagtctgatggcctgagggggctctaccagggtttcaacgtctctgtccaaggcatcattatctatagagctgcctacttcggagtctatgatactgccaaggggatgctgcctgaccccaagaacgtgcacatttttgtgagctggatgattgcccagagtgtgacggcagtcgcagggctgctgtcctacccctttgacactgttcgtcgtagaatgatgatgcagtccggccggaaaggggccgatattatgtacacggggacagttgactgctggaggaagattgcaaaagacgaaggagccaaggccttcttcaaaggtgcctggtccaatgtgctgagaggcatgggcggtgcttttgtattggtgttgtatgatgagatcaaaaaatatgtctaa

SEQ ID NO: 20 - DNA, 897 bp, Artificial sequence
  Human
atgacagatgccgctgtgtccttcgccaaggacttcctggcaggtggagtggccgcagccatctccaagacggcggtagcgcccatcgagcgggtcaagctgctgctgcaggtgcagcatgccagcaagcagatcactgcagataagcaatacaaaggcattatagactgcgtggtccgtattcccaaggagcagggagttctgtccttctggcgcggtaacctggccaatgtcatcagatacttccccacccaggctcttaacttcgccttcaaagataaatacaagcagatcttcctgggtggtgtggacaagagaacccagttttggcgctactttgcagggaatctggcatcgggtggtgccgcaggggccacatccctgtgttttgtgtaccctcttgattttgcccgtacccgtctagcagctgatgtgggtaaagctggagctgaaagggaattccgaggcctcggtgactgcctggttaagatctacaaatctgatgggattaagggcctgtaccaaggctttaacgtgtctgtgcagggtattatcatctaccgagccgcctacttcggtatctatgacactgcaaagggaatgcttccggatcccaagaacactcacatcgtcatcagctggatgatcgcacagactgtcactgctgttgccgggttgacttcctatccatttgacaccgttcgccgccgcatgatgatgcagtcagggcgcaaaggaactgacatcatgtacacaggcacgcttgactgctggcggaagattgctcgtgatgaaggaggcaaagcttttttcaagggtgcatggtccaatgttctcagaggcatgggtggtgcttttgtgcttgtcttgtatgatgaaatcaagaagtacacataa

SEQ ID NO: 21 - DNA, 897 bp, Artificial sequence
  Human
atgacggaacaggccatctccttcgccaaagacttcttggccggaggcatcgccgccgccatctccaagacggccgtggctccgatcgagcgggtcaagctgctgctgcaggtccagcacgccagcaagcagatcgccgccgacaagcagtacaagggcatcgtggactgcattgtccgcatccccaaggagcagggcgtgctgtccttctggaggggcaaccttgccaacgtcattcgctacttccccactcaagccctcaacttcgccttcaaggataagtacaagcagatcttcctggggggcgtggacaagcacacgcagttctggaggtactttgcgggcaacctggcctccggcggtgcggccggcgcgacctccctctgcttcgtgtacccgctggatttcgccagaacccgcctggcagcggacgtgggaaagtcaggcacagagcgcgagttccgaggcctgggagactgcctggtgaagatcaccaagtccgacggcatccggggcctgtaccagggcttcagtgtctccgtgcagggcatcatcatctaccgggcggcctacttcggcgtgtacgatacggccaagggcatgctccccgaccccaagaacacgcacatcgtggtgagctggatgatcgcgcagaccgtgacggccgtggccggcgtggtgtcctaccccttcgacacggtgcggcggcgcatgatgatgcagtccgggcgcaaaggagctgacatcatgtacacgggcaccgtcgactgttggaggaagatcttcagagatgaggggggcaaggccttcttcaagggtgcgtggtccaacgtcctgcggggcatggggggcgccttcgtgctggtcctgtacgacgagctcaagaaggtgatctaa

SEQ ID NO: 22 - DNA, 30 bp, Artificial sequence
  Human
atgggtgatcacgcttggagcttcctaaag

SEQ ID NO: 23 - DNA, 29 bp, Artificial sequence
  Human
ttagacatatttttgatctcatcatacaa

SEQ ID NO: 24 - DNA, 30 bp, Artificial sequence
  Human
atgacagatgccgctgtgtccttcgccaag

SEQ ID NO: 25 - DNA, 30 bp, Artificial sequence
  Human
ttatgtgtacttcttgatttcatcatacaa

SEQ ID NO: 26 - DNA, 30 bp, Artificial sequence
  Human
atgacggaacaggccatctccttcgccaaa

SEQ ID NO: 27 - DNA, 30 bp, Artificial sequence
  Human
ttagatcaccttcttgagctcgtcgtacag

SEQ ID NO: 28 - DNA, 28 bp, Artificial sequence
  Human
taaggtaccatgggtgatcacgcttgga

SEQ ID NO: 29 - DNA, 26 bp, Artificial sequence
  Human
atctcgaggacatattttttgatctc

SEQ ID NO: 30 - DNA, 28 bp, Artificial sequence
  Human
taaggtaccatgacagatgccgctgtgt

SEQ ID NO: 31 - DNA, 26 bp, Artificial sequence
  Human
atctcgagtgtgtacttcttgatttc

SEQ ID NO: 32 - DNA, 28 bp, Artificial sequence
  Human
taaggtaccatgacggaacaggccatct

SEQ ID NO: 33 - DNA, 26 bp, Artificial sequence
  Human
atctcgtggatcaccttcttgagctc